(19) United States
(12) Patent Application Publication
(10) Pub. No.: US 2025/0282929 A1
(43) Pub. Date: Sep. 11, 2025

(54) UNSATURATED ESTERS CONTAINING AN ADDITIVE FOR REDUCING AND STABILIZING THE YELLOWNESS INDEX

(71) Applicant: Röhm GmbH, Darmstadt (DE)

(72) Inventors: Florian Zschunke, Frankfurt (DE); Andrea WIttKowski, Gross-Umstadt (DE); Belaid Ait Aissa, Darmstadt (DE); Rudolf Burghardt, Darmstadt (DE); Daniel Helmut König, Richmond, TX (US); Steffen Krill, Muehltal (DE); Andreas Rühling, Darmstadt (DE); Sven Balk, Darmstadt (DE); Bruno Keller, Wackernheim (DE)

(73) Assignee: Röhm GmbH, Darmstadt (DE)

(21) Appl. No.: 18/559,757

(22) PCT Filed: Apr. 29, 2022
(86) PCT No.: PCT/EP2022/061442

§ 371 Date: Nov. 8, 2023

(30) Foreign Application Priority Data
May 10, 2021 (EP) 21172931.4

(51) Int. Cl. C08K 5/07; C08F 20/14; C08K 5/13; C08K 5/132

(52) U.S. Cl. CPC C08K 5/07; C08F 20/14; C08K 5/13; C08K 5/132; C08K 2201/014

(57) ABSTRACT

A method for reducing the yellowness index of alkyl (meth)acrylates, especially of MMA, and also of polymers which have been produced from these alkyl (meth)acrylates. The method displays this effect even after a relatively long period of storage of monomers. The method involves the addition of specific aldehydes to the monomer composition. This can be done independently of the respective process for preparing the alkyl (meth)acrylates and is therefore simple and inexpensive to implement. The corresponding monomer compositions furthermore form part of the present invention.

### CROSS-REFERENCE TO RELATED APPLICATIONS

This application is a National Stage entry under § 371 of International Application No. PCT/EP2022/061442, filed on Apr. 29, 2022, and which claims the benefit of priority to European Patent Application No. 21172931.4, filed on May 10, 2021. The content of each of these applications is hereby incorporated by reference in its entirety.

### BACKGROUND OF THE INVENTION

### Field of the Invention

The present invention relates to a novel method for reducing the yellowness index of alkyl (meth)acrylates, especially of MMA, and also of polymers which have been produced from these alkyl (meth)acrylates. The novel method displays this effect even after a relatively long period of storage of the monomers. The method involves the addition of specific aldehydes to the monomer composition. This can be done independently of the respective process for preparing the alkyl (meth)acrylates and is therefore simple and inexpensive to implement.

The corresponding monomer compositions furthermore form part of the present invention.

### Description of Related Art

Methyl methacrylate (MMA) is prepared nowadays by various methods proceeding from C2, C3 or C4 units, more predominantly proceeding from hydrogen cyanide and acetone via the acetone cyanohydrin (ACH) formed as central intermediate. This process has the disadvantage that very large amounts of ammonium sulfate are obtained, the processing of which is associated with very high costs. Further processes which use a raw material basis other than ACH are described in the relevant patent literature and in the meantime have been realized on a production scale. A further disadvantage is that the C3-based MMA prepared does not have optimal yellowness indices. Although these are relatively low, they still result in disruptive slight yellow colourations in particular when producing PMMA sheets, films or mouldings which are used in optically relevant applications.

A process for preparing MMA based on C-4 raw materials starts from reactants such as isobutylene or tert-butanol, which over a plurality of process stages are converted into the desired methacrylic acid derivatives. In this case, in a first stage these are oxidized to methacrolein and in a second stage are oxidized to methacrylic acid. Lastly, esterification is effected to give the desired alkyl ester, in particular with methanol to give MMA. More details on this process are given, inter alia, in Ullmann's Encyclopedia of Industrial Chemistry 2012, Wiley-VCH Verlag GmbH & Co. KGaA, Weinheim, Methacrylic Acid and Derivatives, DOI: 10.1002/14356007.a16_441 pub2 and also in Krill and Rühling et al. “Viele Wege führen zum Methacrylsäuremethylester” [Many Routes lead to Methyl Methacrylate], WILEY-VCH Verlag GmbH & Co KGaA, Weinheim, doi.org/10.1002/ciuz.201900869.

Here, isobutylene or tert-butanol is generally oxidized to methacrolein in a first stage, and this methacrolein is then reacted with oxygen to give methacrylic acid. The methacrylic acid obtained is then converted to MMA with methanol. More details on this process are given, inter alia, in Ullmann's Encyclopedia of Industrial Chemistry 2012, Wiley-VCH Verlag GmbH & Co. KGaA, Weinheim, Methacrylic Acid and Derivatives, DOI: 10.1002/14356007.a16_441.pub2.

Specifically, a distinction is made between three processes for preparing MMA on this basis. Raw materials used, for example, are tert-butanol, which is converted to isobutene by elimination of water, or alternatively methyl tert-butyl ether, which is converted to isobutene by elimination of methanol, or isobutene itself, which for example is available as a raw material from a cracker. In summary, this gives rise to the following three routes:

Process A, “tandem C₄ direct oxidation” process, without intermediate isolation of methacrolein: Here, in a first step, methacrolein is prepared from isobutene and is oxidized in a step 2 to methacrylic acid, before this is finally esterified in a step 3 with methanol to give MMA

Process B, “separate C₄ direct oxidation” process: This is identical insofar as in a first step methacrolein is prepared from isobutene, and in a step 2 is first isolated and subjected to intermediate purification, before it is oxidized in a step 3 to methacrylic acid and finally esterified in a step 4 with methanol to give MMA.

Process C, “direct metha process” or direct oxidative esterification process: Here too, in a first step, methacrolein is prepared from isobutene and here too in a step 2 is first isolated and subjected to intermediate purification, before it is directly oxidatively esterified in a step 3 with methanol and air to give MMA.

All the processes described are well documented in the prior art, for example in (i) IHS Chemical Process Economics Program, Review 2015-05, R. J. Chang, Syed Naqvi (ii) Vapor Phase Catalytic Oxidation of Isobutene to Methacrylic Acid, Stud. Surf. Sci. Catal. 1981, 7, 755-767.

Even though the reactant and by-product profile of the C4-based MMA differs appreciably from that obtained proceeding from C3 units, in C4-based MMA without very complex and multistage purification with loss of product a slight, but nevertheless disruptive for optical end applications, yellow colouration is likewise detected. Fundamentally, however, although disruptive, the yellow colouration in C4-based products tends to be somewhat less than that in C3-based products. This yellow colouration was able to be somewhat further reduced, albeit insufficiently, by the provision of an alternative C4-based process.

In this alternative C4-based process, MMA is obtained by gas-phase oxidation of isobutylene or tert-butanol with atmospheric oxygen over a heterogeneous catalyst to afford methacrolein and subsequent oxidative esterification reaction of methacrolein using methanol. This process, developed by ASAHI, is described, inter alia, in publications U.S. Pat. Nos. 5,969,178 and 7,012,039. A particular disadvantage of this process is the very high energy requirement. In a development of the process, the methacrolein is obtained from propanal and formaldehyde in the first stage. Such a process is described in WO 2014/170223. However, even with this optimization it is often found that C4-based MMA often also has appreciable residual yellowness indices.

As an alternative to this process, U.S. Pat. No. 5,969,178 discloses workup in only one column, wherein in said column it is imperative that the feed be situated above the column bottom. Low-boiling constituents from the reactor output are removed from this column overhead. Remaining in the column bottom is a mixture of crude MMA and water, which is to be sent to a further workup. Via a sidestream, the exact position of which must first be determined and can be adjusted by addition of various sieve trays, a mixture of methacrolein and methanol intended for recycling into the reactor is finally withdrawn from the column. U.S. Pat. No. 5,969,178 itself indicates that such a process is difficult to perform on account of a variety of azeotropes. Furthermore, methacrylic acid in particular, which is always present as a by-product, plays an important role. According to this process, despite the silence of U.S. Pat. No. 5,969,178 on this issue, the methacrylic acid would be removed in a manner such that it remains in a phase to be sent for disposal and an isolation would be of only limited worth. However, this means that there is a fall in the overall yield of methacrylic products of this process.

U.S. Pat. No. 7,012,039 discloses a workup of the reactor output from the oxidative esterification which is somewhat of a departure. Here, in a first distillation stage, methacrolein is distilled off overhead via sieve trays and the aqueous, MMA-containing mixture from the bottom is passed into a phase separator. In said phase separator the mixture is adjusted to a pH of about 2 to 3 by addition of sulfuric acid. The separation of the sulfuric-acid-acidified water from the organic/oil phase is then effected by means of centrifuging. This oil phase is separated in a further distillation into high-boiling constituents and an MMA-containing phase which is taken off overhead. The MMA-containing phase is then separated from low-boiling constituents in a third distillation. This is followed by a further fourth distillation for final purification.

The problem with this process is the sulfuric acid which needs to be added in large amounts and can have corrosive effects on parts of the plant. Accordingly, these parts, such as in particular the phase separator or else the second distillation column, have to be fabricated from suitable materials. Moreover, U.S. Pat. No. 7,012,039 is silent regarding the handling of the simultaneously generated methacrylic acid or the residual methanol remaining in the product. However, it can be assumed that the former is also removed in the distillation stages, while the methanol can only partly be obtained and returned with the methacrolein, while the remainder is probably lost in the third distillation stage.

WO 2014/170223 describes a similar process to U.S. Pat. No. 7,012,039. The only difference is that in the actual reaction the pH is adjusted in a circuit by addition of a methanolic sodium hydroxide solution. This serves, inter alia, to protect the catalyst. Moreover, the removal of the aqueous phase in the phase separation is simpler on account of the salt content. However, another consequence is that the methacrylic acid formed is in part in the form of sodium salt and is later removed and disposed of with the aqueous phase. In the variant with sulfuric acid addition in the phase separation, the free acid is indeed recovered, but sodium (hydrogen)sulfate is obtained in return, which can lead to other problems in the disposal.

Lastly, WO 2017/046110 teaches an optimized workup of the crude MMA obtained from an oxidative esterification is first separated from a heavy phase, and an alcohol-containing light phase is then distilled off from this heavy phase and can be recycled in turn. The special feature of this process is moreover that the methacrolein here has been obtained on the basis of propanal and formaldehyde, where the former is obtained on the basis of C2 units, for example from ethylene and synthesis gas.

Overall, all of these processes, irrespective of the raw material basis for the methacrolein used, lead to MMA or in general to alkyl methacrylates, which themselves as monomers exhibit a measurable yellow colouration.

As illustrated in the prior art, the various MMA processes, irrespective of the raw material basis, involve proceeding through a multiplicity of separation steps in order firstly to conduct the isolation of the monomer in accordance with specifications and secondly to achieve a sufficiently low colour number of the monomeric end product. Ultimately transparent polymeric products can be produced in this way.

The slight yellow colouration of the monomers moreover generally increases during a relatively long period of storage, for instance in a storage tank, or as a result of the transport time for the purposes of further processing. The slight yellow colouration of the monomers also leads to a yellow colouration of the downstream products, such as for example moulding compounds or other polymers, for example plexiglass-based pellets and semifinished products produced starting from MMA.

There is therefore a need for improvement in such a way as to identify the source of this yellow colouration and to remove it as efficiently as possible from the corresponding alkyl methacrylate, in particular MMA, prior to the polymerization.

EP 36 762 41 for reducing the yellowness index explicitly proposes adjusting the pH and the water content in a specific manner during the oxidative esterification and further treating the crude product from this stage in a further reactor, where the water content during the aftertreatment is higher and the pH is lower than in the original reaction. While this procedure has proven to be effective, it is also complex in terms of process technology.

As a third raw material alternative there are moreover C2-based processes for preparing alkyl methacrylates, in particular MMA. These processes also include, as intermediate, methacrolein, prepared from formaldehyde and propanal, the latter being obtained from ethylene. In this preparation of methacrolein by the C2 process, the target product is obtained from formalin and propionaldehyde in the presence of a secondary amine and an acid, usually an organic acid. In this case, the reaction is effected via a Mannich reaction. The methacrolein (MAL) synthesized in this way can then be converted in a subsequent step to methacrylic acid by gas-phase oxidation or to methyl methacrylate by oxidative esterification. Such a process for the preparation of methacrolein is described, inter alia, in the publications U.S. Pat. Nos. 7,141,702, 4,408,079, JP 3069420, JP 4173757, EP 0 317 909 and U.S. Pat. No. 2,848,499.

The processes based on a Mannich reaction and suitable for the preparation of methacrolein are generally known to those skilled in the art and are the subject of corresponding review articles, for example in Ullmann's Encyclopedia of Industrial Chemistry 2012, Wiley-VCH Verlag GmbH & Co. KGaA, Weinheim, Acrolein and Methacrolein, DOI: 10.1002/14356007.a01_149.pub2.

For economic exploitation of this process, a high yield and a low specific energy requirement should be attained. The yellow colouration of this C2-based MMA is also lower than for MMA based on alternative raw material sources. Despite this, a yellow colouration, particularly in the polymerized end product, here too can currently be completely avoided only with difficulty and leads, without the addition of bluing agents, to appreciable yellow colourations in colourless, transparent plastics produced from the monomer, such as for example plexiglass sheets.

In summary, it can be said that there are a multiplicity of processes based on the raw materials ethylene, acetone or isobutene. Depending on technologies and workup methods, and also specifically for the various preparation processes, a monomer quality—for example of the MMA—is produced which in actuality can differ on the basis of the trace components.

For the C2-based LIMA process, it can generally be stated that no (meth)acrylonitrile is present in the product, but there is a relatively high content in the ppm range of isobutyric acid methyl ester, which is also referred to as methyl isobutyrate. The content generally varies between 100 and 700 ppm. Other characteristic traces are dimethoxyisobutene and dimethoxyisobutane.

For the likewise C2-based ALPHA process, which just as with the LIMA process is based on ethylene as base material, it can generally be stated that no (meth)acrylonitrile is present here either. However, in its place there may be present a relatively high value in the ppm range of propionic acid methyl ester, which is also referred to as methyl propionate. The content varies between 10 and 100 ppm. In comparison with the LIMA process, there is a reduced presence of methyl isobutyrate (a few tens of ppm). Other characteristic traces in the MMA from the ALPHA process are pentanones, such as for example diethyl ketones or isopropyl methyl ketone, and ethanol.

For the C3-based ACH-sulfo process, which is predominantly based on acetone as starting material, it can essentially be stated that (meth)acrylonitrile is generally present in concentrations from 30 to 250 ppm. Methyl propionate and methyl isobutyrate are likewise detected, but in lower concentrations than in the C2-based processes. Pentanones, such as diethyl ketone or isopropyl methyl ketone, and ethanol are not found, or can be found only in the single-digit ppm range.

The C4-based processes, in particular those conducted as gas-phase processes, in turn comprise other specific traces. Here too, traces of methyl isobutyrate and methyl propionate are detected, however in distinction dimethylfuran and pyruvic acid are in particular present as trace components which also have an influence on the yellowness index in the isolated monomer.

As a specific C4-based process, the Asahi process must be highlighted at this juncture, this process including a direct oxidative liquid-phase oxidation as second reaction step. With this MMA quality, methyl isobutyrate is again found as characteristic trace component.

In most of the processes, especially in the two C3- and C4-based processes, diacetyl is a colour-imparting component that has to be removed in the isolation process but which partly makes its way into the isolated MMA. As a result, contents of between just over 0 and 10 ppm can be detected in commercially available MMA.

Taking into consideration this specific composition of the MMA monomer qualities produced by the various processes, it was a particularly complex task to reduce the yellow colouration of the products and to counteract the subsequent yellowing during transport and storage.

Overall, there is therefore a great need for effectively and simply counteracting the yellow colouration of MMA. In particular, this need exists irrespective of the process for MMA preparation.

### SUMMARY OF THE INVENTION

### Problems

Therefore, a problem addressed by the present invention was that of reducing the yellowness index of alkyl (meth)acrylates, in particular of MMA, in the simplest possible way.

A particular problem here was that this reduction should be attainable irrespective of the preparation method of the alkyl (meth)acrylate.

A further problem was that the reduction in the yellowness index should be lastingly present, i.e. even after a long period of storage of the composition containing the alkyl (meth)acrylate.

A further problem was that of providing a monomeric product quality of the alkyl (meth)acrylate which is improved in terms of the yellowness index. In this connection, the problem arose that this improved optical product quality of the monomers should also lead, after the polymerization to give the poly(meth)acrylates thus produced, to improved optical properties having a reduced yellowness index.

Furthermore, the method for lasting yellowness index reduction should be toxicologically harmless and simple and inexpensive to use.

A further problem was that the method should be implementable without relatively major modifications to the production plants and without relatively major investment.

Further problems which are not stated explicitly may become apparent from the description, subject matter as described herein, the examples, or the overall context of the present description of the invention.

### Solution

The problems have been solved by means of a novel method for reducing the yellowness index of alkyl (meth)acrylates. This method is characterized in that 0.5 to 500 ppm by weight of an aldehyde having the general formula R—HC═O are added to the alkyl (meth)acrylate. Surprisingly, the aldehydes are relatively freely choosable. For instance, according to the invention aldehydes are usable which have a radical R having between 1 and 20 carbon atoms and optionally up to three oxygen atoms as ether and/or hydroxy groups. Here, R can be a linear, branched or cyclic alkyl group, an aromatic group, an ether group or a combination of two or more of these groups.

Examples of common linear alkyl groups are ethyl, propyl, n-butyl, n-hexyl or n-dodecyl groups. Branched alkyl groups include those alkyl groups having one or more e.g. tertiary or quaternary carbon atoms Examples of these are isopropyl, iso- or tert-butyl or ethylhexyl groups. Cyclic alkyl groups may for example be cyclohexyl, cyclopentyl or methylcyclohexyl groups.

Besides saturated alkyl groups, aromatic groups or combinations of aromatic and saturated alkyl groups are also usable Examples of aromatic groups are phenyl or benzyl groups

According to the invention, radicals may furthermore be used which contain a total of up to 20 carbon atoms and additional oxygen atoms in the form of one or more ether or hydroxy groups.

Aldehydes containing olefinic groups are not usable in accordance with the invention since they are potentially polymerization-active. In addition, they do not appear to display any effect, as can be determined from different concentrations of residual methacrolein in C2- or C4-MMA.

In addition, other heteroatoms, such as in particular nitrogen or sulfur heteroatoms, in the aldehyde are excluded since these may be subject to oxidation sensitivity, for example, and may themselves in turn lead to discolouration. Halogen atoms are in turn unsuitable for reasons of reactivity and from a toxicological viewpoint.

The aldehyde is particularly preferably acetaldehyde, propanal, 3-methylpentanal, iso- or n-butanal and n-pentanal.

The present method is particularly preferably used for the additivation of commercially customary alkyl (meth)acrylates such as methyl methacrylate (MMA). However, other monomers, such as in particular n- or tert-butyl methacrylate, ethylhexyl methacrylate, ethyl methacrylate or propyl methacrylate, may also be additivated. The method is additionally usable for acrylates such as methyl or butyl acrylate. The yellowness indices of important functional (meth)acrylates, such as methacrylic acid, hydroxyethyl (meth)acrylate or hydroxypropyl (meth)acrylate, may also be reduced. The alkyl (meth)acrylate is preferably methyl methacrylate.

### BRIEF DESCRIPTION OF THE DRAWINGS

FIG. 1 compares the results of Examples 1 to 12, in comparison to Comparative Examples CE1, 2 and 3, in terms of a stabilization of C2-, C3- and C4-MMA with various concentrations of isobutanal (see also results in Table 1).

FIG. 2 compares the results of Examples 13 to 16 of a stabilization of C3-MMA over a period of 8 weeks of storage (at 50° C.) with various concentrations of isobutanal, in comparison to Comparative Example CE4 (see also results in Table 2).

FIG. 3 compares the results of Examples 17 to 20 of a stabilization of C4-MMA over a period of 8 weeks of storage (at 50° C.) with various concentrations of isobutanal, in comparison to Comparative Example CE5 (see also results in Table 2).

### DETAILED DESCRIPTION OF THE INVENTION

According to the invention, between 0.5 and 500 ppm by weight of the aldehyde are added to the respective monomer composition. The optimal amount depends here on the (meth)acrylate to be additivated and on the aldehyde used. This amount can be determined for the respective combination by a person skilled in the art by means of a few simple manual experiments. For many of these combinations, a preferred added aldehyde amount of between 1 and 250 ppm by weight, especially preferably between 10 and 150 ppm by weight, has proven to be advantageous.

Preference is given to additionally adding between 1 and 300 ppm by weight of one or more polymerization stabilizers to the alkyl (meth)acrylate. Preference is given to using only one polymerization stabilizer. Polymerization stabilizers for (meth)acrylates are known in general to those skilled in the art. Preference is given to using 2,4-dimethyl-6-tert-butylphenol (DMBP) or a hydroquinone, very particularly preferably hydroquinone methyl ether (HQME), in combination with the method according to the invention.

Preferably, the method according to the invention is implemented in such a way that one hour after addition of the aldehyde the alkyl (meth)acrylate has a yellowness index [D65/10] that is reduced by at least 10%, particularly preferably at least 15%. Particularly preferably, one hour after addition of the aldehyde, such as e.g. isobutanal, the alkyl (meth)acrylate has a yellowness index [D65/10] that is reduced by at least 40%.

Surprisingly it has been found that the yellowness index of an alkyl (meth)acrylate not only can be strikingly lowered within a short time by simple addition of the aldehydes described. At least equally surprisingly, it has been found that this reduction in the yellowness index is lasting such that it is still detectable to the same or at least a similar degree even after storage for a plurality of days. This can be observed even after storage at elevated temperatures, such as for example at 40° C. Preferably, the method according to the invention is implemented here in such a way that 8 days, preferably 1 month, after addition of the aldehyde the alkyl (meth)acrylate still has a yellowness index [D65/10] that is reduced by at least 10%, particularly preferably at least 15%. No or only a very slight increase in the yellowness index of the composition is generally detected over this period—compared to the yellowness index one hour after addition of the aldehyde.

Very surprisingly, it has furthermore been found that the yellowness index of polymers produced from alkyl (meth)acrylates additivated in accordance with the invention is also markedly reduced compared to analogously produced polymers without the additivation according to the invention. This effect is still stable even after a long period of storage of the polymers, for example for one month. The colour stabilization is readily measurable and surprisingly strong, even after weathering tests of the polymers.

In principle, the method according to the invention is usable not just for reducing the yellowness index of pure alkyl (meth)acrylates such as MMA, but also of monomer mixtures predominantly consisting of various alkyl (meth)acrylates. In this case, the aldehyde may be added to the monomer mixture, or alternatively one or more aldehydes have already been added in accordance with the invention to an admixed monomer such that the overall mixture is obtained with an inventive concentration of the aldehyde.

The effect of the invention also exists in the polymers produced from these monomer mixtures.

Surprisingly, it has also been found that many, more precisely all, investigated aldehydes that correspond to the description above display the effect of the invention. According to tests performed, for example, methanal, acetaldehyde, propanal, iso- or n-butanal, pentanal, 2-methylpentanal, decanal, dodecanal, are particularly well suited.

Aromatic aldehydes, such as benzaldehyde, 3-hydroxybenzaldehyde, also display an effect, albeit an initially reduced effect compared to aldehydes with purely alkyl groups. These are therefore usable in accordance with the invention, but are less preferred.

In addition to the method according to the invention, compositions containing at least 97.5% by weight of an alkyl (meth)acrylate also form part of the present invention. These compositions are characterized according to the invention in that they contain between 0.5 and 500 ppm by weight of an aldehyde having the general formula R—HC═O. The same applies for the aldehyde as stated above in the context of the method. Particularly preferred aldehydes according to the invention are isobutanal, n-pentanal or 3-methylpentanal.

Particularly preferably—but in a non-restrictive fashion—the alkyl (meth)acrylate is methyl methacrylate (MMA). The composition in this case preferably contains at least 99.5% by weight, ideally at least 99.9% by weight, of MMA. Further monomers that in accordance with the invention may be present in the composition have already been identified in the description with respect to the method.

According to the invention, the composition preferably includes at least 97.5% by weight of the alkyl (meth)acrylate and at least between 0.5 and 500 ppm by weight of the aldehyde. Preferably, the composition contains 99.5% by weight, particularly preferably 99.8% by weight, of alkyl (meth)acrylate and includes between 1 and 300 ppm by weight, especially between 20 and 250 ppm by weight and very particularly preferably between 10 and 130 ppm by weight, especially between 30 and 90 ppm by weight, of the aldehyde. The aldehyde/aldehydes in the composition is/are particularly preferably methanal, acetaldehyde, propanal, iso- or n-butanal, pentanal, 2-methylpentanal, decanal, dodecanal, or a mixture of at least two of these aldehydes.

The composition according to the invention preferably additionally contains between 1 and 300 ppm by weight of a polymerization stabilizer. This is preferably 2,4-dimethyl-6-tert-butylphenol or a hydroquinone, very particularly preferably hydroquinone methyl ether (HQME).

Surprisingly, it has moreover been found that, by use of the method according to the invention or using the compositions according to the invention, a colour stabilization of functional or non-functional alkyl (meth)acrylates, especially of the—commercially most significant—MMA, can be achieved independently of the particular underlying preparation processes. However, it was particularly surprising here that the effect of the invention arises for MMA fundamentally independently of the preparation process, but with respect to the extent of the effect, in particular in relation to the degree of colour reduction, very much exhibits a dependence on the underlying preparation process. This can empirically only be attributed to interactions with other components in the particular compositions. Nevertheless, it could in no way be expected that the effect might be detected despite very different secondary components in the alkyl (meth)acrylate.

For instance, the effect of the invention is particularly strongly pronounced in MMA, or other alkyl (meth)acrylates, which inter alia have been prepared by means of the C3-based ACH process. This surprising effect can, according to analyses, be attributed in particular to the fact that the composition contains acrylonitrile and/or methacrylonitrile. It is particularly preferable here when in total less than 300 ppm by weight, in particular less than 200 ppm by weight, of acrylonitrile and methacrylonitrile are present in the composition.

The effect of the invention is likewise particularly strongly pronounced in MMA, or other alkyl (meth)acrylates, which inter alia have been prepared by means of a C2-based process. This surprising effect can, according to analyses, be attributed in particular to the fact that the composition contains at least two components selected from n-butanol, tert-butanol, methyl acrylate, methyl isobutyrate, methyl propionate, 1,1-dimethoxyisobutene and ethyl methacrylate, especially when the composition includes n-butanol, tert-butanol, methyl acrylate, methyl propionate and ethyl methacrylate. It is particularly preferable here when all of these components are present, but where in total less than 5 ppm by weight of n-butanol, tert-butanol, methyl acrylate, methyl propionate and ethyl methacrylate are present in the composition. It is equally preferable when in total less than 700 ppm by weight of n-butanol, tert-butanol, methyl isobutyrate, methyl acrylate, methyl propionate and ethyl methacrylate are present.

Likewise pronounced, albeit not as markedly as in the other cases, is the effect of the invention in MMA, or other alkyl (meth)acrylates, which inter alia have been prepared by means of a C4-based process proceeding from isobutene, isobutanol or MTBE.

This surprising effect can, according to analyses, be attributed in particular to the fact that the composition contains dimethylfuran, methyl pyruvate and/or diacetyl, and preferably all three components. It is particularly preferable here when in total less than 30 ppm by weight, in particular less than 10 ppm by weight, of these three components are present in the composition.

### EXAMPLES

### Reduction in the Yellowness Index

In order to determine the reduction in the yellowness index of methyl methacrylate from the C2, C3 or C4 process as a result of the addition of the aldehyde isobutanal in accordance with the present invention, methyl methacrylate was doped with an aldehyde, such as for example isobutanal. This procedure relates initially to Examples 1 to 12. Then, and/or at specified points in time, the yellowness index Y.I. D65/10° was determined in accordance with DIN 6167. The following raw materials were used to prepare the doped methyl methacrylate samples:
methyl methacrylate from a C2 process from Röhm, prepared by the LiMA process (hereinafter C2-MMA)
methyl methacrylate from a C3 process from Röhm, prepared by the ACH process (hereinafter C3-MMA)
methyl methacrylate from a C4 process from Röhm, prepared from isobutene, which had already been stabilized with 50 ppm of hydroquinone monomethyl ether (hereinafter C4-MMA)
isobutanal from Merck KGaA

To prepare the methyl methacrylate samples doped with isobutanal for investigation, methyl methacrylate was initially charged in a glass beaker, if required the stabilizer hydroquinone monomethyl ether was dissolved therein and isobutanal was added. The mixture was homogenized for one hour with a magnetic stirrer. The yellowness index was then determined to assess the optical quality.

### Comparative Example CE1 (Reference Example for Yellowness Index)

C3-MMA, stabilized with 50 ppm of hydroquinone monomethyl ether, without addition of an aldehyde

### Example 1

C3-MMA, stabilized with 50 ppm of hydroquinone monomethyl ether and admixed with 12 ppm of isobutanal

### Example 2

C3-MMA, stabilized with 50 ppm of hydroquinone monomethyl ether and admixed with 25 ppm of isobutanal

### Example 3

C3-MMA, stabilized with 50 ppm of hydroquinone monomethyl ether and admixed with 60 ppm of isobutanal

### Example 4

C3-MMA, stabilized with 50 ppm of hydroquinone monomethyl ether and admixed with 100 ppm of isobutanal

### Comparative Example CE2 (Reference Example)

C4-MMA, already containing 50 ppm of hydroquinone monomethyl ether, without addition of an aldehyde

### Example 5

C4-MMA, already containing 50 ppm of hydroquinone monomethyl ether, admixed with 12 ppm of isobutanal

### Example 6

C4-MMA, already containing 50 ppm of hydroquinone monomethyl ether, with 25 ppm of isobutanal

### Example 7

C4-MMA, already containing 50 ppm of hydroquinone monomethyl ether, admixed with 60 ppm of isobutanal

### Example 8

C4-MMA, already containing 50 ppm of hydroquinone monomethyl ether, admixed with 100 ppm of isobutanal

### Comparative Example CE3 (Reference Example)

C2-MMA, already containing 50 ppm of hydroquinone monomethyl ether, without addition of an aldehyde

### Example 9

C2-MMA, already containing 50 ppm of hydroquinone monomethyl ether, admixed with 12 ppm of isobutanal

### Example 10

C2-MMA, already containing 50 ppm of hydroquinone monomethyl ether, with 25 ppm of isobutanal

### Example 11

C2-MMA, already containing 50 ppm of hydroquinone monomethyl ether, admixed with 60 ppm of isobutanal

### Example 12

C2-MMA, already containing 50 ppm of hydroquinone monomethyl ether, admixed with 100 ppm of isobutanal

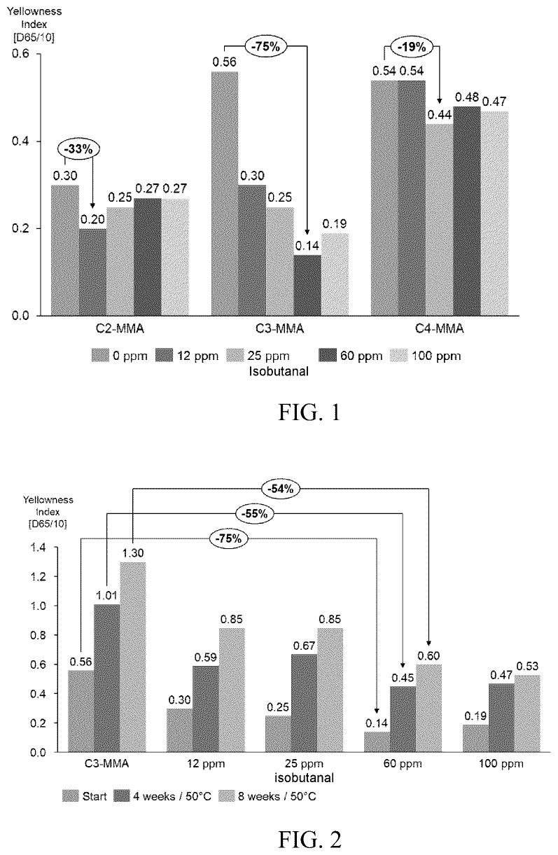

The respective yellowness indices of the isobutanal-doped methyl methacrylate samples of the C3-MMA, C4-MMA and C2-MMA, respectively, were related to the respective yellowness index of the pure methyl methacrylate from the C3, C4 and C2 process, respectively (CE1, CE2 and CE3). This yields the percentage reduction in the yellowness index compared to the starting value. The values are presented in Table 1 and are visually compared in FIG. 1.

TABLE 1

| | | | | Percentage change |
|---|---|---|---|---|
| | | | Yellowness | compared to |
| | | Isobutanal | index | starting value |
| Example | MMA | [ppm] | D65/10 | [%] |
| CE1 | C3-MMA | 0 | 0.56 | |
| 1 | C3-MMA | 12 | 0.30 | −46.4% |
| 2 | C3-MMA | 25 | 0.25 | −55.4% |
| 3 | C3-MMA | 60 | 0.14 | −75.0% |
| 4 | C3-MMA | 100 | 0.19 | −66.1% |
| CE2 | C4-MMA | 0 | 0.54 | |
| 5 | C4-MMA | 12 | 0.53 | −1.9% |
| 6 | C4-MMA | 25 | 0.44 | −18.5% |
| 7 | C4-MMA | 60 | 0.48 | −11.1% |
| 8 | C4-MMA | 100 | 0.47 | −13.0% |
| CE3 | C2-MMA | 0 | 0.30 | |
| 9 | C2-MMA | 12 | 0.20 | −33.3% |
| 10 | C2-MMA | 25 | 0.25 | −16.7% |
| 11 | C2-MMA | 60 | 0.27 | −10.0% |
| 12 | C2-MMA | 100 | 0.27 | −10.0% |

### Assessment of the Reduction in the Yellowness Index Over Time

In order to demonstrate a stable reduction in the yellowness index over a relatively long period of time, samples of C3- and C4-MMA according to the present invention were admixed with isobutanal as aldehyde and stored at 50° C. Corresponding experiments can be found in Examples 9 to 16 and the associated Comparative Examples CE4 and CE5. The yellowness index Y.I. D65/10° was determined in accordance with DIN 6167 according to the points in time specified in Table 2. The same raw materials were used as in Examples 1 to 12.

To prepare the samples doped with isobutanal for investigation, methyl methacrylate was initially charged in a glass beaker, if required the stabilizer hydroquinone monomethyl ether was dissolved therein and isobutanal was added. The mixture was homogenized for one hour with a magnetic stirrer 25±1 g of the solution were then filled into brown 30 ml narrow-neck bottles and stored at 50° C. in an air circulation drying cabinet.

To assess the optical quality, the yellowness index was determined at the start of storage at 50° C. and after a storage time of 4 weeks and 8 weeks at a corresponding storage temperature of 50° C.

### Comparative Example CE4 (Reference Example for Yellowness Index after Storage)

C3-MMA, stabilized with 50 ppm of hydroquinone monomethyl ether, without addition of an aldehyde
with storage at 50° C. for 8 weeks in an air circulation drying cabinet

### Example 13

C3-MMA, stabilized with 50 ppm of hydroquinone monomethyl ether and admixed with 12 ppm of isobutanal
and storage at 50° C. for 8 weeks in an air circulation drying cabinet

### Example 14

C3-MMA, stabilized with 50 ppm of hydroquinone monomethyl ether and admixed with 25 ppm of isobutanal
and storage at 50° C. for 8 weeks in an air circulation drying cabinet

### Example 15

C3-MMA, stabilized with 50 ppm of hydroquinone monomethyl ether and admixed with 60 ppm of isobutanal
and storage at 50° C. for 8 weeks in an air circulation drying cabinet

### Example 16

C3-MMA, stabilized with 50 ppm of hydroquinone monomethyl ether and admixed with 100 ppm of isobutanal and storage at 50° C. for 8 weeks in an air circulation drying cabinet

### Comparative Example CE5 (Reference Example)

C4-MMA, already containing 50 ppm of hydroquinone monomethyl ether without addition of an aldehyde and with storage at 50° C. for 8 weeks in an air circulation drying cabinet

### Example 17

C4-MMA, already containing 50 ppm of hydroquinone monomethyl ether and admixed with 12 ppm of isobutanal and storage at 50° C. for 8 weeks in an air circulation drying cabinet

### Example 18

C4-MMA, already containing 50 ppm of hydroquinone monomethyl ether and admixed with 25 ppm of isobutanal and storage at 50° C. for 8 weeks in an air circulation drying cabinet

### Example 19

C4-MMA, already containing 50 ppm of hydroquinone monomethyl ether and admixed with 60 ppm of isobutanal and storage at 50° C. for 8 weeks in an air circulation drying cabinet

### Example 20

C4-MMA, already containing 50 ppm of hydroquinone monomethyl ether and admixed with 100 ppm of isobutanal and storage at 50° C. for 8 weeks in an air circulation drying cabinet

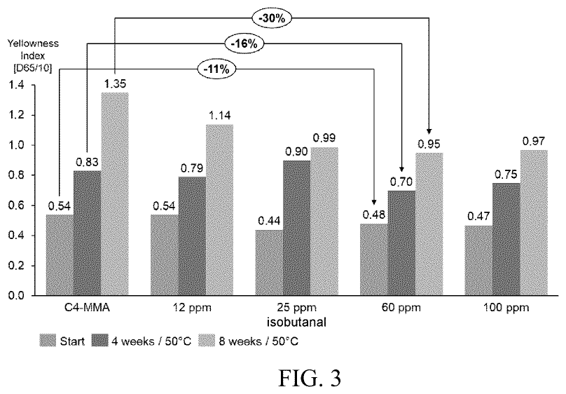

The respective yellowness indices of the isobutanal-doped methyl methacrylate samples of the C3-MMA and C4-MMA, respectively, were in each case related, without storage and also after storage for 4 or 8 weeks, to the respective yellowness index of the pure methyl methacrylate from the C3 and C4 process, respectively. This yields the percentage reduction in the yellowness index compared to the starting value. The values can be found in Table 2 and, with specification of the percentage reduction, are presented graphically in FIG. 2 for C3-MMA and FIG. 3 for C4-MMA.

TABLE 2

| | | | Yellowness index [D65/10] | | |
|---|---|---|---|---|---|
| | | Isobutanal | Storage time at 50° C. | | |
| Example | MMA | [ppm] | 0 weeks | 4 weeks | 8 weeks |
| CE4 | C3 process | 0 | 0.56 | 1.01 | 1.30 |
| 13 | C3 process | 12 | 0.30 | 0.59 | 0.85 |
| 14 | C3 process | 25 | 0.25 | 0.67 | 0.85 |
| 15 | C3 process | 60 | 0.14 | 0.45 | 0.68 |
| 16 | C3 process | 100 | 0.19 | 0.47 | 0.53 |
| CE5 | C4 process | 0 | 0.54 | 0.83 | 1.35 |
| 17 | C4 process | 12 | 0.54 | 0.79 | 1.14 |
| 18 | C4 process | 25 | 0.44 | 0.90 | 0.99 |
| 19 | C4 process | 60 | 0.48 | 0.70 | 0.95 |
| 20 | C4 process | 100 | 0.47 | 0.75 | 0.97 |

### Comparative Example CE1 (Reference Example)

C3-MMA stabilized with 50 ppm of hydroquinone monomethyl ether, without addition of an aldehyde

### Examples 21 to 34

C3-MMA stabilized with 50 ppm of hydroquinone monomethyl ether, admixed with an aldehyde as indicated in Table 3 and storage at 50° C. for 8 weeks in an air circulation drying cabinet, with measurements of the yellowness index after 4 weeks and 8 weeks The results can be found in Table 3.

Observation concerning Example 27: the measurement of the yellowness index after the direct addition of 10 ppm by weight of dodecanal appears to be attributable to a measurement error. The reduction in the yellowness index after 4/8 weeks is also consistent in this example with the other examples and the results to be expected in accordance with the invention.

### Examples 35 to 40

C3-MMA stabilized with 50 ppm of hydroquinone monomethyl ether, admixed with n-pentanal as indicated in Table 4 and storage at 50° C. for 8 weeks in an air circulation drying cabinet, with measurements of the yellowness index after 4 weeks and 8 weeks The results can be found in Table 4.

TABLE 3
Yellowness indices and percentage reduction compared to starting value of C3-MMA

| | | | | Percentage | | Percentage | | Percentage |
|---|---|---|---|---|---|---|---|---|
| | | | Yellowness | change in | Yellowness | change in | Yellowness | change in |
| | | | index | yellowness | index | yellowness | index | yellowness |
| | Aldehyde | Conc. | D65/10 | index | D65/10 | index | D65/10 | index |
| Example | added | [ppm] | Initial | | 4 weeks at 50° C. | | 8 weeks at 50° C. | |
| CE1 | — | 0 | 0.91 | | 1.12 | | 1.29 | |
| 21 | methanal | 10 | 0.81 | −11% | 0.83 | −26% | 1.04 | −19% |
| 22 | methanal | 100 | 0.58 | −36% | 0.78 | −30% | 1.32 | 2% |
| 23 | propanal | 10 | 0.85 | −7% | 1.04 | −7% | 1.06 | −18% |
| 24 | propanal | 100 | 0.78 | −14% | 0.95 | −15% | 1.09 | −16% |
| 25 | decanal | 10 | 0.85 | −7% | 0.78 | −30% | 1.01 | −22% |
| 26 | decanal | 100 | 0.62 | −32% | 0.8 | −29% | 1.00 | −22% |
| 27 | dodecanal | 10 | 1.33 | 46% | 1.03 | −8% | 1.26 | −2% |
| 28 | dodecanal | 100 | 0.71 | −22% | 1.09 | −3% | 1.34 | 4% |
| 29 | 3-hydroxybenzaldehyde | 10 | 0.56 | −38% | 0.78 | −30% | 1.32 | 2% |
| 30 | 3-hydroxybenzaldehyde | 100 | 0.85 | −7% | 0.83 | −26% | 1.01 | −22% |
| 31 | 4-hydroxybenzaldehyde | 10 | 0.64 | −30% | 1.35 | 21% | 1.42 | 10% |
| 32 | 4-hydroxybenzaldehyde | 100 | 0.83 | −9% | 1.15 | 3% | 1.51 | 17% |
| 33 | 3,4,5-trimethoxy- | 10 | 0.64 | −30% | 0.77 | −31% | 1.06 | −18% |
| | benzaldehyde | | | | | | | |
| 34 | 3,4,5-trimethoxy- | 100 | 0.61 | 33% | 0.98 | −13% | 1.15 | −11% |
| | benzaldehyde | | | | | | | |

TABLE 4
Yellowness indices and percentage reduction compared to starting value
of C3-MMA (acetone-based MMA process) with addition of n-pentanal

| | | | | Percentage | | Percentage | | Percentage |
|---|---|---|---|---|---|---|---|---|
| | | | Yellowness | change | Yellowness | change | Yellowness | change |
| | | Concen- | index | compared to | index | compared to | index | compared to |
| | | tration | D65/10 | starting value | D65/10 | starting value | D65/10 | starting value |
| Example | | [ppm] | Initial | | 4 weeks at 50° C. | | 8 weeks at 50° C. | |
| CE1 | — | 0 | 0.56 | | 1.01 | | 1.30 | |
| 35 | n-pentanal | 1 | 0.26 | −54% | 0.60 | −41% | 0.60 | −54% |
| 36 | n-pentanal | 5 | 0.35 | −38% | 0.28 | −72% | 0.60 | −54% |
| 37 | n-pentanal | 10 | 0.20 | −64% | 0.27 | −73% | 0.45 | −65% |
| 38 | n-pentanal | 25 | 0.17 | −70% | 0.53 | −48% | 0.73 | −44% |
| 39 | n-pentanal | 50 | 0.25 | −55% | 0.76 | −25% | 0.58 | −55% |
| 40 | n-pentanal | 100 | 0.24 | −57% | 0.48 | −52% | 0.59 | −55% |

1. A method for reducing the yellowness index of alkyl (meth)acrylates, comprising:
adding 0.5 to 500 ppm by weight of an aldehyde having a formula R—HC═O to an alkyl (meth)acrylate,
wherein R has between 1 and 20 carbon atoms and optionally up to three oxygen atoms as ether and/or hydroxy groups and R is a linear, branched, or cyclic alkyl group, an aromatic group, an ether group, or a combination of two or more of these groups.

2. The method according to claim 1, wherein the alkyl (meth)acrylate is methyl methacrylate.

3. The method according to claim 1, wherein between 1 and 150 ppm by weight of the aldehyde are added to the alkyl (meth)acrylate.

4. The method according to claim 1, wherein between 1 and 300 ppm by weight of one or more polymerization stabilizers are additionally added to the alkyl (meth)acrylate.

5. The method according to claim 1, wherein 1 h after addition of the aldehyde, the alkyl (meth)acrylate has a yellowness index [D65/10] that is reduced by at least 5%.

6. The method according to claim 5, wherein 1 h after addition of the aldehyde, the alkyl (meth)acrylate has a yellowness index [D65/10] that is reduced by at least 15%.

7. The method according to claim 1, wherein the aldehyde is at least one selected from the group consisting of methanal, acetaldehyde, propanal, iso- or n-butanal, pentanal, 2-methylpentanal, decanal, dodecanal, benzaldehyde, aid 3-hydroxybenzaldehyde.

8. A composition containing:
at least 97.5% by weight of an alkyl (meth)acrylate, between 0.5 and 500 ppm by weight of an aldehyde having a formula R—HC═O,
wherein R has between 1 and 20 carbon atoms and optionally up to three oxygen atoms as ether and/or hydroxy groups and R is a linear, branched, or cyclic alkyl group, an ether group, an aromatic group, or a combination of two or more of these groups.

9. The composition according to claim 8, wherein the alkyl (meth)acrylate is methyl methacrylate (MMA), and wherein the composition contains at least 99.5% by weight of MMA.

10. The composition according to claim 8, wherein the composition includes at least 99.8% by weight of the alkyl (meth)acrylate and between 1 and 250 ppm by weight of the aldehyde.

11. The composition according to claim 10, wherein the composition includes between 10 and 130 ppm by weight of the aldehyde.

12. The composition according to claim 8, wherein the aldehyde is selected from the group consisting of methanal, acetaldehyde, propanal, iso- or n-butanal, pentanal, 2-methylpentanal, decanal, dodecanal, and a mixture of at least two of these aldehydes.

13. The composition according to claim 8, wherein the composition contains acrylonitrile and/or methacrylonitrile, and wherein in total less than 200 ppm by weight of acrylonitrile and methacrylonitrile are present in the composition.

14. The composition according to claim 8, wherein the composition contains dimethylfuran, methyl pyruvate, and diacetyl, and wherein in total less than 30 ppm by weight of dimethylfuran, diacetyl, and methyl pyruvate are present in the composition.

15. The composition according to claim 8, wherein the composition contains at least two components selected from the group consisting of n-butanol, tert-butanol, methyl isobutyrate, methyl acrylate, 1,1-dimethoxyisobutene, methyl propionate and ethyl methacrylate, and wherein in total less than 700 ppm by weight of n-butanol, tert-butanol, methyl isobutyrate, methyl acrylate, 1,1-dimethoxyisobutene, methyl propionate, and ethyl methacrylate are present.

16. The composition according to claim 8, further comprising between 1 and 300 ppm by weight of a polymerization stabilizer.

17. The method according to claim 4, wherein the one or more polymerization stabilizers are DMBP (2,4-dimethyl-6-tert-butylphenol) or HQME (hydroquinone monomethyl ether).

18. The composition according to claim 16, wherein the polymerization stabilizer is HQME (hydroquinone monomethyl ether).
